(19) United States
(12) Patent Application Publication
(10) Pub. No.: US 2010/0311106 A1
(43) Pub. Date: Dec. 9, 2010

(54) QUANTITATION OF LOBULAR INVOLUTION FOR BREAST CANCER RISK PREDICTION

(76) Inventors: Lynn C. Hartmann, Rochester, MN (US); Carol Reynolds, Rochester, MN (US); Kevin P. McKian, Rochester, MN (US)

Correspondence Address: FISH & RICHARDSON P.C. (TC), PO BOX 1022, MINNEAPOLIS, MN, 55440-1022, US

(21) Appl. No.: 12/863,889

(22) PCT Filed: Jan. 21, 2009
(86) PCT No.: PCT/US09/31560

§ 371 Date: Aug. 20, 2010

Related U.S. Application Data
(62) Division of application No. 61/023,586, filed on Jan. 25, 2008.

(51) Int. Cl. C12Q 1/02

(52) U.S. Cl. 435/29

(57) ABSTRACT

Methods for determining risk of developing breast cancer are described.

### CROSS-REFERENCE TO RELATED APPLICATIONS

This application claims priority from U.S. Provisional Application Ser. No. 61/023,586, filed Jan. 25, 2008.

### STATEMENT AS TO FEDERALLY SPONSORED RESEARCH

This invention was made with government support under grant no. DAMD 17-02-1-0473, awarded by the Department of Defense. The government has certain rights in the invention.

### TECHNICAL FIELD

This document relates to quantifying lobular involution to determine the risk of developing breast cancer.

### BACKGROUND

The mammary gland undergoes profound physiologic changes throughout the phases of a woman's life, including puberty, pregnancy, lactation, postlactational involution, and aging. The epithelial tissue of the human breast is organized into 15-20 major lobes, each made up of lobules that contain the milk-forming acini. These lobules, also referred to as “terminal duct lobular units,” are the anatomic structures that give rise to breast cancer. As a woman ages, breast lobules regress, or involute, with a reduction in the number and size of acini per lobule and replacement of the delicate intralobular stroma with the more dense collagen of breast connective tissue. Over time, there is progressive fatty replacement of glandular elements and collagen. See Cowan and Herbert, Surg Pathol 1989; 2: 323-333. This process differs greatly from postlactational involution. After lactation, there is regression of all breast tissues as secretory activity is curtailed, but there is no substantive loss of glandular tissue. Russo and Russo, Maturitas 2004; 49:2-15.

Although involution of the breast involves a consistent sequence of histologic changes, the rate and extent of involution vary considerably among individual women. This age-related lobular involution has been documented in women younger than age 40 years and thus involves factors not limited to the onset of menopause. Hutson et al., J Clin Pathol 1985; 38: 281-7.

The extent of age-related involution of terminal duct lobular units in a woman's breast tissue is associated with the subsequent risk of developing breast cancer. Women with “complete involution” (75-100% of lobules demonstrating involution) had a decreased risk of developing breast cancer. Alternatively, women with “no involution” (0%) were at higher risk. A large group of women were defined as having “partial involution” (1-74%). See Milanese et al., J Natl Cancer Inst 2006; 98:1600-1607. Because the approach was qualitative and had a wide range of involution in the “partial” involution group, a need exists for a quantitative method of assessing lobular involution as a means of risk prediction.

### SUMMARY

Disclosed is a method for determining risk of developing breast cancer by quantifying lobular involution in a subject (e.g., a female subject that has had a benign breast biopsy). For example, lobular area or number of acini per lobule can be quantified in breast tissue. Subjects with a larger lobular area and higher number of acini per lobule are at more risk of developing breast cancer than a subject in which lobular area is smaller or number of acini per lobule is lower in the tissue sample. A lobule area greater than 49000μ² (e.g., greater than 59000μ²) indicates the subject is at higher risk of developing breast cancer. Subjects also are at higher risk of developing breast cancer when the tissue sample contains at least 18 acini per lobule (e.g., at least 21 acini per lobule).

In some embodiments, this document features a method for assessing risk of breast cancer in a woman. The method can include providing a breast tissue sample from the woman; quantifying lobular area in the sample; and classifying the woman as being at an increased risk of developing breast cancer if the lobular area is greater than 49000μ², or classifying the woman as being at a decreased risk of breast cancer if the lobular area is less than 49000μ². The method further can include determining number of acini per lobule in the breast tissue sample, and classifying the woman as being at an increased risk of developing breast cancer if the lobular area is greater than 49000μ² and the number of acini per lobule is at least 18; or classifying the woman as being at a decreased risk of breast cancer if the lobular area is less than 49000μ² and the number of acini per lobule is 17 or less.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and from the claims.

### DETAILED DESCRIPTION

In general, methods and materials are disclosed for determining if a subject has an increased risk of developing breast cancer. Suitable subjects can be mammals, including, for example, humans, non-human primates such as monkeys, baboons, or chimpanzees, horses, cows (or oxen), pigs, sheep, goats, cats, rabbits, guinea pigs, hamsters, rats, gerbils, and mice. Female patients who have had benign breast biopsies are particularly useful subjects. In some embodiments, a subject can be a woman with benign breast disease, which includes atypical hyperplasia, proliferative disease without atypia, nonproliferative disease, fibroadenoma, and other benign breast disease.

Methods for assessing risk of developing breast cancer in a female subject (e.g., a woman that has benign breast disease) can include quantifying lobular involution in a breast tissue sample. For example, a method for assessing a subject's risk of developing breast cancer can include quantifying lobular area and/or number of acini per lobule in the breast tissue sample. A breast tissue sample can be stained (e.g., with hematoxylin and eosin) such that structures within the breast can be visualized with microscopy. Lobular area and number of acini per lobule can be quantified manually or by using software. For example, stained breast tissue slides can be scanned and analyzed using software such as Webslide™ browser software from Bacus Laboratories, Inc. (Lombard, Ill.). See also U.S. Pat. Nos. 6,272,235 and 6,396,941. Lobular area and number of acini per lobule can be compared to that of a control population (e.g., the average lobular area in a group of female subjects with or without breast cancer or the average number of acini per lobule in a group of female subjects with or without breast cancer). Subjects at higher risk for developing breast cancer have a lobular area greater than 49000μ² (e.g., greater than 50000, 51000, 52000, 53000, 54000, 55000, 56000, 57000, 58000, or 59000μ²). Subjects at higher risk for developing breast cancer have at least 18 acini per lobule (e.g., at least 19, 20, 21, or 22 acini per lobule). As such, a subject can be classified as having a decreased risk of developing breast cancer if the lobular area is less than 49000μ² or classified as having an increased risk of developing breast cancer if the lobular area is greater than 49000μ² (e.g., greater than 59000μ²). A subject also can be classified as having a decreased risk of developing breast cancer if the number of acini per lobule is 17 or less or classified as having an increased risk of developing breast cancer if the number of acini per lobule is 18 or more. Additional risk factors can be considered when determining risk of developing breast cancer including, for example, family history and other genetic factors, e.g., mutations within the BRCA1 and BRCA2 genes.

Methods described herein can be implemented in a computer system having a processor that executes specific instructions in a computer program. The computer system may be arranged to output a patient's risk of developing breast cancer based on receiving a patient's lobular area or number of acini per lobule.

The methods provided herein can be used to assist a medical or research professional (e.g., a physician, clinician, technician, nurse, or research scientist). For example, a method can include communicating to a medical or research professional the lobular area or number of acini per lobule in a breast tissue sample from a subject.

The invention will be further described in the following example, which does not limit the scope of the invention described in the claims.

### EXAMPLE

In this example, a nested case control study was performed within the Mayo Benign Breast Disease Cohort (BBD). The BBD database is a collection of breast tissue on all women who had open breast biopsies at Mayo between 1967 and 1991 and who were found to have benign breast disease. Subsequent outcome data has been obtained for all these women. In this nested case-control study, 86 cases and 152 controls were selected for a total number of 238. Approximately two controls to each case were matched to age and time period of biopsy. Hematoxylin and Eosin stained slides were scanned into the computer and analyzed using Webslide™ browser software (Bacus labs product). The 10 largest normal lobules were analyzed for each individual. Analysis included area of the lobule and number of acini per lobule. Mean number of acini and mean lobule area were compared for cases and controls, involution status (none, partial, or complete), histology (NP, PDWA, or AH), and family history (none, weak, or strong).

Women who went on to develop breast cancer had a larger lobular area (59458μ² vs 49221μ²; p=0.0452) and higher number of acini per lobule (21.62 vs. 16.11; p=0.0006) compared with women who did not develop cancer. Women with no involution had a larger lobule area (102013μ²) and number of acini per lobule (35.72) than women with partial (56945μ², 20.85 acini per lobule) or complete involution (27254μ², 8.72 acini per lobule) (p<0.0001). The difference between lobular area and number of acini per lobule was not significantly significant when evaluating for effect of histology or family history (p=0.153 and 0.4770) respectively. Thus, lobular involution can be quantified and may be used as a risk predictor for women who have had benign breast biopsies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining risk of developing breast cancer in a subject, said method comprising:
(a) providing a breast tissue sample from said subject; and
(b) quantifying lobular involution in said sample, wherein a larger lobular area or higher number of acini per lobule in said tissue sample indicates said subject is at increased risk of developing breast cancer than a subject in which lobular area is smaller or number of acini per lobule is lower in said tissue sample.

2. The method of claim 1, wherein a lobule area greater than 49000μ² indicates the subject is at more risk of developing breast cancer.

3. The method of claim 1, wherein a lobule area greater than 59000μ² indicates the subject is at more risk of developing breast cancer.

4. The method of claim 1, wherein at least 18 acini per lobule indicates the subject is at increased risk of developing breast cancer.

5. The method of claim 1, wherein at least 21 acini per lobule indicates the subject is at increased risk of developing breast cancer.

6. The method of claim 1, wherein said subject has had a benign breast biopsy.

7. A method for assessing risk of breast cancer in a woman, said method comprising:
(a) providing a breast tissue sample from said woman;
(b) quantifying lobular area in said sample; and
(c) classifying said woman as being at an increased risk of developing breast cancer if said lobular area is greater than 49000μ², or classifying said woman as being at a decreased risk of breast cancer if said lobular area is less than 49000μ².

8. The method of claim 7, further comprising determining number of acini per lobule in said sample, and classifying said woman as being at an increased risk of developing breast cancer if said lobular area is greater than 49000μ² and said number of acini per lobule is at least 18; or classifying said woman as being at a decreased risk of breast cancer if said lobular area is less than 49000μ² and said number of acini per lobule is 17 or less.
